(19) United States
(12) Patent Application Publication
(10) Pub. No.: US 2017/0258841 A1
(43) Pub. Date: Sep. 14, 2017

(54) PHARMACEUTICAL COMPOSITIONS FOR TREATING ARRHYTHMIA AND THERAPEUTICS OF

(71) Applicant: Gwo Xi Stem Cell Applied Technology Co., Ltd., Zhubei City (TW)

(72) Inventors: Tsung-Ming LEE, Zhubei City (TW); Ming-Hsi CHUANG, Zhubei City (TW); Chun-Hung CHEN, Zhubei City (TW); Po-Cheng LIN, Zhubei City (TW); Chia-Hsin Lee, Zhubei City (TW)

(21) Appl. No.: 15/448,050

(22) Filed: Mar. 2, 2017

Related U.S. Application Data
(60) Provisional application No. 62/306,888, filed on Mar. 11, 2016.

(51) Int. Cl. A61K 35/28; A61K 9/00; A61K 31/5377; A61K 33/00; A61K 31/404

(52) U.S. Cl. CPC A61K 35/28; A61K 33/00; A61K 31/404; A61K 31/5377; A61K 9/0019

(57) ABSTRACT

This invention provides a pharmaceutical compositions for treating arrhythmia. The pharmaceutical compositions comprise stem cells, pretreated with n-butylidenephthalide (BP).

### CROSS-REFERENCE TO RELATED APPLICATIONS

This Non-provisional application claims priority under 35 U.S.C. §119(a) on Patent Application No(s). [62/306,888] filed in United States America [Mar. 11, 2016], the entire contents of which are hereby incorporated by reference.

### FIELD OF THE INVENTION

This invention provides a pharmaceutical compositions for treating arrhythmia, wherein the pharmaceutical compositions comprise stem cells.

### BACKGROUND OF THE INVENTION

Cardiac arrhythmia, also known as cardiac dysrhythmia or irregular heartbeat, is a group of conditions in which the heartbeat is irregular, too fast, or too slow. A heartbeat that is too fast—above 100 beats per minute in adults—is called tachycardia and a heartbeat that is too slow—below 60 beats per minute—is called bradycardia. Many types of arrhythmia have no symptoms. When symptoms are present these may include palpitations or feeling a pause between heartbeats. More seriously there may be lightheadedness, passing out, shortness of breath, or chest pain. While most types of arrhythmia are not serious, some predispose a person to complications such as stroke or heart failure. Others may result in cardiac arrest.

There are four main types of arrhythmia: extra beats, supraventricular tachycardias, ventricular arrhythmias, and bradyarrhythmias. Extra beats include premature atrial contractions and premature ventricular contractions. Supraventricular tachycardias include atrial fibrillation, atrial flutter, and paroxysmal supraventricular tachycardia. Ventricular arrhythmias include ventricular fibrillation and ventricular tachycardia. Arrhythmias are due to problems with the electrical conduction system of the heart. Arrhythmias may occur in children; however, the normal range for the heart rate is different and depends on age. A number of tests can help with diagnosis including an electrocardiogram (ECG) and holter monitor.

Most arrhythmias can be effectively treated. Treatments may include medications, medical procedures such as a pacemaker, stem cells, and surgery. Medications for a fast heart rate may include beta blockers or agents that attempt to restore a normal heart rhythm such as procainamide. This later group may have more significant side effects especially if taken for a long period of time. Pacemakers are often used for slow heart rates. Those with an irregular heartbeat are often treated with blood thinners to reduce the risk of complications. Those who have severe symptoms from an arrhythmia may be treated emergently with a jolt of electricity in the form of cardioversion or defibrillation.

Although the treatments for arrhythmia developed raiply, but all of these treatment would bring some sequela.

### SUMMARY OF THE INVENTION

According to the defect of the treatments, this invention providing a pharmaceutical compositions for treating arrhythmia.

In some embodiments, the pharmaceutical compositions for treating arrhythmia comprise a stem cell.

In some embodiments, the stem cells is pre-treated with a n-butylidenephthalide (BP) and an inhibitor.

In some embodiments, the inhibitor is a GSK-3β inhibitor or PI3K inhibitor.

In some embodiments, the GSK-3β inhibitor or the PI3K inhibitor is Lithium.

In some embodiments, the GSK-3β inhibitor is SB216763.

In some embodiments, the stem cell is an adipose-derived stem cell.

In some embodiments, the n-butylidenephthalide (BP) concenration range is from 7 μg/ml to 40 μg/ml.

In some embodiments, the GSK-3β inhibitor(LITHIUM) concenration range is from 0.3 mM to 3 mM.

In some embodiments, the PI3K inhibitor concenration range is 20 μM for LY294002.

A method for therapeutic arrhythmia in a subject, wherein the method comprising administering to said subject an effective amount of a pharmaceutical composition as claim 1.

In some embodiments, the effective amount concentration range is from 1×10⁶ to 1×10⁷ cells.

In some embodiments, the pharmaceutical composition is administering to said subject by intra-myocardial [symbol] intra-cardiac [symbol] intra-coronary artery [symbol] intra-cornary vein [symbol] trans-endocardial•intravenous [symbol] intra-muscular [symbol] intra-dermal [symbol] sub-cutaneous [symbol] intra-peritioneal [symbol] intra-pleural•systemic injection.

In some embodiments, the arrhythmia is cause by Myocardial infarction.

The details of one or more embodiments of the invention are set forth in the description below. Other features or advantages of the present invention will be apparent from the following detailed description of an example and also from the appended claims.

### BRIEF DESCRIPTION OF THE DRAWINGS

FIGS. 1A, 1A-1, 1B, 1C, 1C-1. Transplantation of ADSCs pretreated with BP reduces fibrosis and increases angiogenesis. (FIG. 1A, FIG. 1A-1) Sirius red staining. (FIG. 1A) Representative images and (FIG. 1A-1) quantification of fibrotic area in the remote zone by Sirius red staining at 4 weeks post-MI. (A represents sham, B represents vehicle, C represents ADSC, D represent ADSC-BP, E represent ADSC-BP-Li, F represent ADSC-BP-SB, G represent ADSC-BP-LY in FIG. 1A) (FIG. 1B) Hydroxyproline measurement from the samples from the remote zone at 4 weeks post-MI. (C)(FIG. 1C, FIG. 1C-1) α-SMA staining. Representative images and (FIG. 1C-1) quantification of arteriole density in the border zone at 4 weeks post-MI analyzed by α-SMA staining. Scale bar, 50 μm. *p<0.05 compared with sham; †p<0.05 compared with the infarcted group treated with vehicle; ‡p<0.05 compared with infarcted groups treated with ADSCs and ADSCs/BP/LY. (A represents vehicle, B represents ADSC, C represents ADSC-BP, D represent ADSC-BP-Li, E represent ADSC-BP-SB, F represent ADSC-BP-LY in FIG. 1C)

FIGS. 2A, 2B, 2B-1. Myocardial (FIG. 2 A) superoxide and (FIG. 2B, FIG. 2B-1) DHE staining from the remote zone. (FIG. 2B)Representative images and (FIG. 2B-1) quantification. (n=5 each group). *p<0.005 compared with sham; †<0.05 compared with infarcted groups treated with vehicle and ADSCs/BP/LY; ‡p<0.05 compared with infarcted group treated with ADSCs. (A represents sham, B represents vehicle, C represents ADSC, D represent ADSC-BP, E represent ADSC-BP-Li, F represent ADSC-BP-SB, G represent ADSC-BP-LY in FIG. 2B)

FIGS. 3A, 3A-1, 3B, 3B-1. Immunofluorescent staining for tyrosine hydroxylase and growth associated protein 43 from the remote regions (magnification 400×). (FIG. 3A, FIG. 3A-1) (fluorescein isothiocyanate stain), tyrosine hydroxylase. (FIG. 3A) Representative images and (FIG. 3A-1) quantification. Tyrosine hydroxylase-positive nerve fibers are located between myofibrils and are oriented longitudinal direction as that of the myofibrils. (A represents sham, B represents vehicle, C represents ADSC, D represent ADSC-BP, E represent ADSC-BP-Li, F represent ADSC-BP-SB, G represent ADSC-BP-LY in FIG. 3A) (FIG. 3B, FIG. 3B-1) (rhodamine stain), growth associated protein 43. (FIG. 3B)Representative images and (FIG. 3B-1) quantification. n=5 each group. Bar=50 μm. Each column and bar represents mean ±SD. *p<0.005 compared with sham; †p<0.05 compared with infarcted groups treated with vehicle and ADSCs/BP/LY; ‡p<0.05 compared with infarcted group treated with ADSCs. (A represents sham, B represents vehicle, C represents ADSC, D represent ADSC-BP, E represent ADSC-BP-Li, F represent ADSC-BP-SB, G represent ADSC-BP-LY in FIG. 3B)

FIG. 4. Inducibility quotient of ventricular arrhythmias by programmed electrical stimulation 4 weeks after MI in an in vivo model (n=5 each group). *p<0.005 compared with sham; †<0.05 compared with infarcted groups treated with vehicle and ADSCs/BP/LY; ‡p<0.05 compared with infarcted group treated with ADSCs.

FIGS. 5A, 5B, 5C, 5D. (FIG. 5A)Western blot showed that BP primed ADSCs resulted in a significant increase (p<0.05) in relative p-Akt level compared with that in ADSCs alone. (FIG. 5B)Western blot showed that NGF levels were significantly upregulated 2.45-fold in the remote zone in the vehicle-treated infarcted rats than in sham-operated rats (p<0.001). (FIG. 5C) PCR amplification of the cDNA revealed that the NGF mRNA levels showed a 2.25-fold upregulation in the remote zone in the vehicle-treated infarcted rats compared with sham-operated rats (p<0.001). (D) (FIG. 5D) To further elucidate the physiological effect of attenuated sympathetic hyperinnervation, ventricular pacing was performed. Arrhythmia score in sham-operated rats was very low (0.2+0.1).

### DETAILED DESCRIPTION OF THE INVENTION

Methods

Chemicals

BP (A10353) was purchased from Alfa Aesar. BP was dissolved in dimethylsulfoxide (DMSO; Sigma), incubated with shaking at 25° C. for 1 h, and stored at 4° C. until use.

Isolation of Human ADSCs

Human ADSCs were generously provided by Gwo Xi Stem Cell Applied Technology (Hsinchu, Taiwan) according to a previously described method after approval by the Institution Review Board of the China Medical University. In brief, this human ADSC line was derived from female donors who underwent gynecological surgery. Passaged cultures were deemed passage 1. ADSCs at passages 3-5 were used in this study. These ADSCs were homogeneous and did not contain endothelial cells or hematopoietic lineages. Cultured ADSCs have been shown to display mesenchymal stem cell phenotype: they express the mesenchymal stem cell marker CD90 and do not express hematopoietic markers CD31 and CD34. The cell quality has been approved by the authority in Taiwan. Besides, the hADSCs have been used in clinical trial for liver cirrhosis.

The cells were maintained in Iscove's modified Dulbecco's medium (Invitrogen-Gibco) supplemented with 10% (v/v) FBS, 10 ng/ml bFGF (R&D Systems, Minneapolis, Minn.), 2 mM L-glutamine, and 100 U/L penicillin—streptomycin (Invitrogen-Gibco). For experiments, ADSCs were thawed and incubated in a 37° C. incubator with 5% CO2.

Animals

Animal experiments were approved and conducted in accordance with local institutional guidelines for the care and use of laboratory animals at the China Medical University and conformed with the Guide for the Care and Use of Laboratory Animals published by the US National Institutes of Health (NIH Publication No. 85-23, revised 1996).

Induction of MI and Cell Transplantation

Male Wistar rats (250-300 g) were subjected to ligation of the anterior descending artery, resulting in infarction of the LV free wall as we previously described For surgery, hemodynamics measurements, electrophysiological studies and sacrifice, rats were intraperitoneally anaesthetized with ketamine (90 mg/kg body weight) and xylazine (9 mg/kg). Anesthesia monitor was tested by rear foot reflexes before and during procedures, observation of respiratory pattern, and responsiveness to manipulations throughout the procedures. Animals were ventilated with 95% O₂ and 5% CO₂ using a ventilator (Harvard Apparatus 486).

Twenty-four hours after ligation, rats were randomly assigned into groups of either control or cell transplantation. For cell transplantation, ADSCs were detached from the plate, suspended in 30 μl of PBS (1×10⁶ cells), and transplanted at six injection sites into the viable myocardium bordering the infarction using a syringe with a 30-gauge needle. ADSC were primed with either 100 μg/ml BP, BP+3 mM lithium (a GSK-3β inhibitor), or BP+10 μM SB216763 (3-(2,4-dichlorophenyl)-4-(1-methyl-1H-indol-3-yl)-1Hpyrrole-2,5-dione, a selective ATP competitive GSK-3α/β antagonist), or BP+20 μM LY294002 (a PI3K inhibitor) for 3 hours before transplantation.

Lithium was used as a positive control as it is known that lithium pretreatment attenuated infarct volumes in an experimentally-induced stroke, by inhibiting GSK-3 signaling. Lithiums is both the GSK-3β and P13K agonist inhibitor and the SB216763 is the GSK-3β inhibitor.

The doses of BP, LiCl, SB216763, and LY294002 were used as previously described. The incubation period was designed to be 3 hours because previous studies have shown that Nurr77 mRNA was significantly induced 30 min after BP treatment. Before cell transplantation, cells were washed for 3 times to eliminate the direct drug effects. The study duration was 4 weeks so as to extend beyond the majority of the myocardial remodeling process in the rat (70-80%) which is complete within 3 weeks. Sham operation served as controls. Thus, together, the experimental groups studied will be: sham group and infarction groups (vehicle, ADSC, BP-ADSC, (BP/lithium)-ADSC, (BP/SB216763)-ADSC, and (BP/LY294002)-ADSC).

Hemodynamics and Infarct Size Measurements

Hemodynamic parameters were measured in anesthetized rats at the end of the echocardiogram. A polyethylene Millar catheter was inserted into the LV and connected to a transducer (Model SPR-407; Miller Instruments, Houston, Tex., USA) to measure LV systolic and diastolic pressure as the mean of measurements of five consecutive pressure cycles as previously described. The maximal rates of LV pressure rise (+dP/dt) and decrease (−dP/dt) were measured. After the arterial pressure measurement, the electrophysiological tests were performed. At completion of the electrophysiological tests, the atria and the right ventricle were trimmed off, and the LV was rinsed in cold physiological saline, weighed, and immediately frozen in liquid nitrogen after a coronal section of the LV was obtained for infarct size estimation. A section, taken from the equator of the LV, was fixed in 10% formalin and embedded in paraffin for determination of infarct size. Each section was stained with hematoxylin and eosin and trichrome. The infarct size was determined as previously described.

In vivo Electrophysiological Studies

To assess the potential arrhythmogenic risk, we performed in vivo programmed electrical stimulation after left thoracotomy and artificial respiration. Because the residual neural integrity at the infarct site is one of the determinants of the response to electrical induction of ventricular arrhythmias, only rats with transmural scar were included. Body temperature was kept at 37° C. with a thermostatically controlled heating lamp. Programmed electrical stimulation was performed with electrodes sewn to the epicardial surface of the right-ventricular outflow tract. Pacing pulses were generated from a Bloom stimulator (Fischer Imaging Corp., Denver, Colo., USA). To induce ventricular arrhythmias, pacing was performed at a cycle length of 120 ms (S1) for 8 beats, followed by one to three extrastimuli (S2, S3, and S4) at shorter coupling intervals. The endpoint of ventricular pacing was induction of ventricular tachyarrhythmia. Ventricular tachyarrhythmias including ventricular tachycardia and ventricular fibrillation were considered nonsustained when they lasted <15 beats and sustained when they lasted >15 beats. A modified arrhythmia scoring system was used, as previously described. When multiple forms of arrhythmias occurred in one heart, the highest score was used. The experimental protocols were typically completed within 10 min.

Morphometry of Cardiac Fibrosis

Aniline blue and picrosirius staining, a collagen-specific stain (Sirius Red F3BA; Pfaltz & Bauer, Stamford, Conn.), were used to stain 5-μm thick, paraffin-embedded sections in the remote area (>2 mm within the infarct). The interstitial collagen fraction was determined by quantitative morphometry of the picrosirius-stained sections with an automated image analyzer (Image Pro Plus, CA). These parameters were assessed in a blinded fashion by at least two investigators. The density of labeled areas was qualitatively estimated from 10 randomly selected fields at a magnification of 400×. The value was expressed as the ratio of the labeled area to total area.

Immunohistochemistry (α-SMA, Tyrosine Hydroxylase, Growth Associated Protein 43, Neurofilament)

Tissue samples from the border and remote zones were frozen using 2-methylbutane, then embedded in optimal cutting temperature compound (Tissue-Tek, Torrance), and sectioned at 5 μm on a microtome. The slides were washed with PBTx (0.1% Triton X-100 in PBS), blocked with 1% BSA (Amresco), and 1.5% normal goat serum (Vector laboratories) in PBT× at 37° C. for 1 h. Then the sections were incubated at 4° C. overnight with a primary antibody to anti-α-smooth muscle cell actin (α-SMA) (1:100; ab5694; Abcam), anti-tyrosine hydroxylase (1:200; Chemicon, Calif., USA), anti-growth associated protein 43 (a marker of nerve sprouting, 1:400; Chemicon, Calif., USA), and anti-neurofilament antibodies (a marker of sympathetic nerves, 1:1000; Chemicon, Calif., USA) in 0.5% BSA in PBS overnight at 37° C. The second antibody was monoclonal goat anti-mouse IgG conjugated to fluorescein isothiocyanate for tyrosine hydroxylase and rhodamine for growth associated protein 43 and neurofilament. Isotype-identical directly conjugated antibodies served as a negative control.

The slides were coded so that the investigator will be blinded to the identification of the rat sections. The target density was measured on the tracings by computerized planimetry (Image Pro Plus, Media Cybernetics, Silver Spring, Md.) as described previously. The target density was qualitatively estimated from 10 randomly selected fields at a magnification of 400× and expressed as the ratio of labeled target area to total area.

In situ Detection of Superoxide

For evaluating myocardial intracellular superoxide production using in situ dihydroethidium (DHE; Invitrogen Molecular Probes, Eugene, Oreg., USA) fluorescence, OCT-embedded tissues were incubated with DHE as previously described.

Laboratory Measurements

Although cardiac innervation was detected by immunofluorescent staining of tyrosine hydroxylase and growth-associated factor 43, it did not imply that the nerves are functional. Thus, to examine the sympathetic nerve function, we measured LV norepinephrine levels from the remote zone. Total norepinephrine was measured using a commercial ELISA kit (Noradrenalin ELISA, IBL Immuno-Biological Laboratories Co., Hamburg, Germany).

Superoxide production by myocardium from the remote zone was measured using lucigenin (5 μM bis-N-methylacridinium nitrate, Sigma, St. Louis, Mo.) enhanced chemiluminescence as previously described. The specific chemiluminescence signal was calculated after subtraction of background activity and expressed as counts per minute per milligram weight (cpm/mg).

Histological collagen results were confirmed by hydroxyproline assay adapted from Stegemann and Stalder. The samples from the remote area were immediately placed in liquid nitrogen and stored at −80° C. until measurement of the hydroxyproline content. The results were calculated as hydroxyproline content per weight of tissue.

Western Blot Analysis of Aktl and NGF

Samples obtained from the remote zone at week 4 after infarction. Antibodies to p-Akt1 (ser473, Cell Signaling technology), Akt1 (Santa Cruz Biotechnology), and NGF (Chemicon) were used. Western blotting procedures were described previously. Experiments were replicated three times and results expressed as the mean value. Effects of BP-pretreated ADSC transplantation on arrhythmias.

Real-Time RT-PCR of Human Alu and NGF

To follow the fate of human ADSCs, we used real-time PCR assays for human-specific Alu sequences 28 days after infarction. RT-PCR was performed from samples obtained from the border and remote zones with the TaqMan system (Prism 7700 Sequence Detection System, PE Biosystems, Foster City, Calif., USA) as previously described. Primers sequences were the following: human Alu sense 5′-CATGGTGAAACCCCGTCTCTA-3′ (SEQ ID: NO. 1), antisense 5′-GCCTCAGCCTCCCGAGTAG-3′(SEQ ID: NO. 2); NGF sense 5′-CACACTGAGGTGCATAGCGT-3′(SEQ ID: NO. 3), antisense 5′-TGATGACCGCTTGCTCCTGT-3′(SEQ ID: NO. 4); cyclophilin sense 5′-ATGGTCAACCCCACCGTGTTCTTCG-3′ (SEQ ID: NO. 5), antisense 5′-CGTGTGAAGTCACCACCCTGACACA-3′ (SEQ ID: NO. 6). Reaction conditions were programmed on a computer linked to the detector for 40 cycles of the amplification step.

Statistical Analysis

Results were presented as mean±SD. Statistical analysis was performed using the SPSS statistical package (SPSS, version 19.0, Chicago, Ill.). Differences among the groups of rats were tested by an ANOVA. In case of a significant effect, the measurements between the groups were compared with Bonferroni's correction. Electrophysiological data (scoring of programmed electrical stimulation-induced arrhythmias) was compared by a Kruskal-Wallis test followed by a Mann-Whitney test. Probability values were 2-tailed, and a value of P<0.05 was considered significant.

Results

Differences in mortality among the infarcted groups were not found throughout the study (Table 1). Relative heart weights normalized by body weight at the end of the experimental period (12 weeks of age) are not significant differences among groups. Four weeks after infarction, the infarcted area of the LV was very thin and was totally replaced by fully differentiated scar tissue. The weight of the LV inclusive of the septum remained essentially constant 4 weeks among the infarcted groups. The lung weight/body weight ratio, an index of lung edema, was significantly lower in the ADSC-, ADSC/BP-, ADSC/BP/Li-, and ADSC/BP/SB216763-treated infarcted groups compared with those in the vehicle- and ADSC/BP/LY294002-treated infarcted groups. The values of +dp/dt and −dp/dt were significantly higher in the ADSC/BP-, ADSC/BP/Li-, and ADSC/BP/SB216763-treated infarcted groups compared with those in the ADSC alone. LV end-systolic pressure, LV end-diastolic pressure, and infarct size did not differ among the infarcted groups.

Effects of BP-Pretreated ADSC Transplantation on Cardiac Function

To determine whether BP generates an enhanced therapeutic effect of stem cells, we evaluated the effects of ADSC transplantation on cardiac function in post-MI rat hearts (Table 2). LV fractional shortening was significantly decreased in the MI group compared with sham. Fractional shortening was significantly improved in the ADSC group. Additional improvement of fractional shortening in cardiac function was observed in the group treated with ADSCs pretreated with BP compared with ADSC alone. However, addition of either lithium or SB216763 did not further improve LV fractional shortening compared with BP-treated group. Conversely, the rats to which LY294002 was administered impaired LV fractional shortening compared with the BP-treated group alone. These results show that ADSCs reduce the post-MI deterioration of cardiac function, and this cardioprotective effect is significantly greater in the BP-treated groups via a PI3K/Akt/GSK-3β dependent pathway.

Effects of BP-Pretreated ADSC Transplantation on Myocardial Fibrosis and Angiogenesis

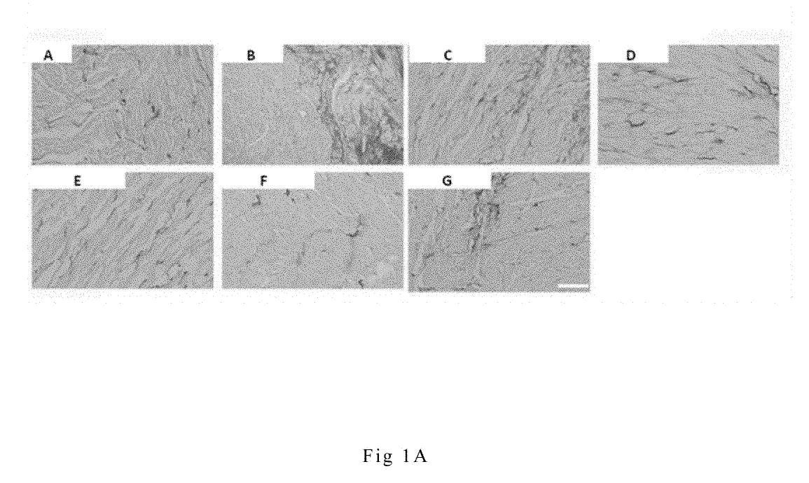

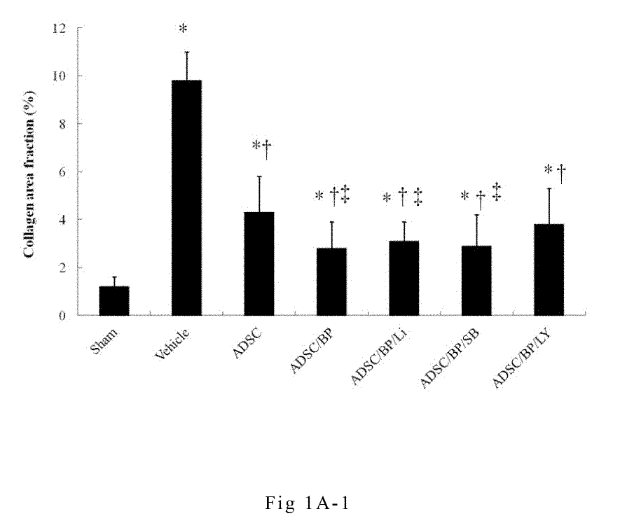

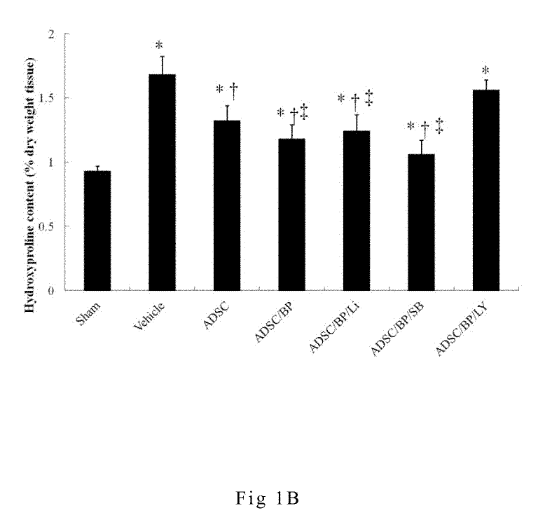

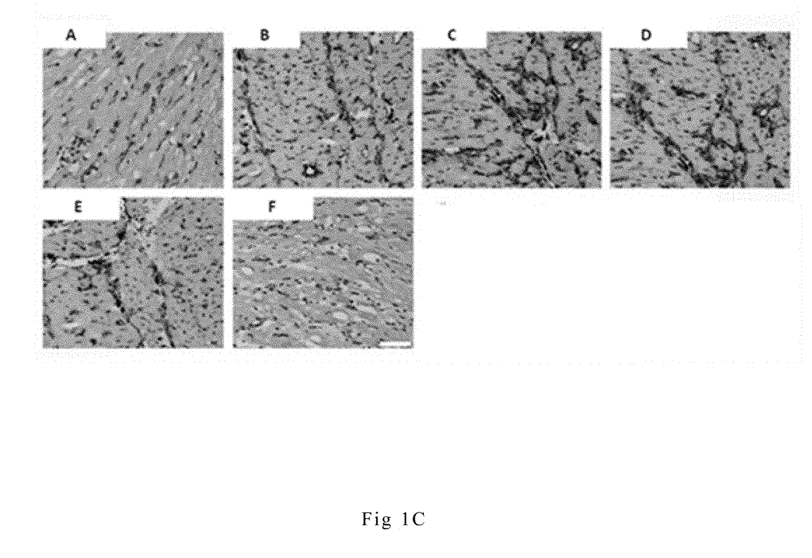

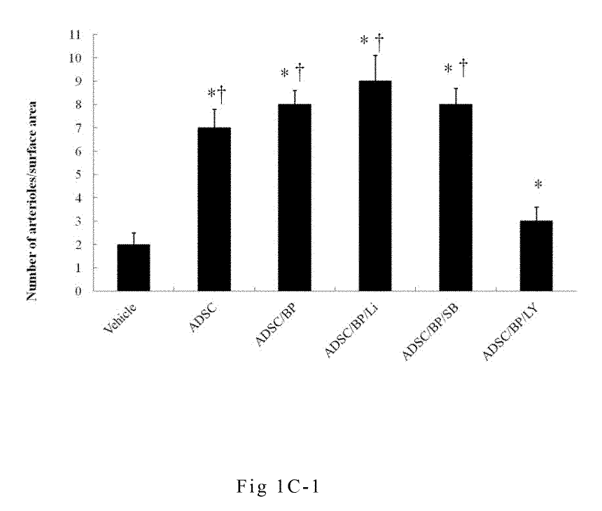

The extent of fibrotic tissue assessed by Sirius red staining and hydroxyproline amount was significantly reduced in the ADSC group compared with the vehicle group (FIGS. 1A, 1A-1, 1B). The hearts that received ADSCs pretreated with BP showed significantly further reduction in fibrosis compared with the ADSC group alone. The ADSCs pretreated with BP can increase the number of stay the enhance the therapeutic effect. Can enhance the effect of ADSC to local organ treatment.

At day 28 after inoculation, we analyzed the total number of arterioles in the border zone using α-SMA staining, respectively (FIGS. 1C, 1C-1). Infarcted rats treated with ADSCs had significantly increased blood vessel density compared with vehicle. These data suggest that the enhanced therapeutic effect of ADSC treatment for MI is associated with reduced fibrosis and increased angiogenesis. Some ADSCs differentiate into cardiomyocytes and can replace their functions.

Effects of BP-Pretreated ADSC Transplantation on ROS

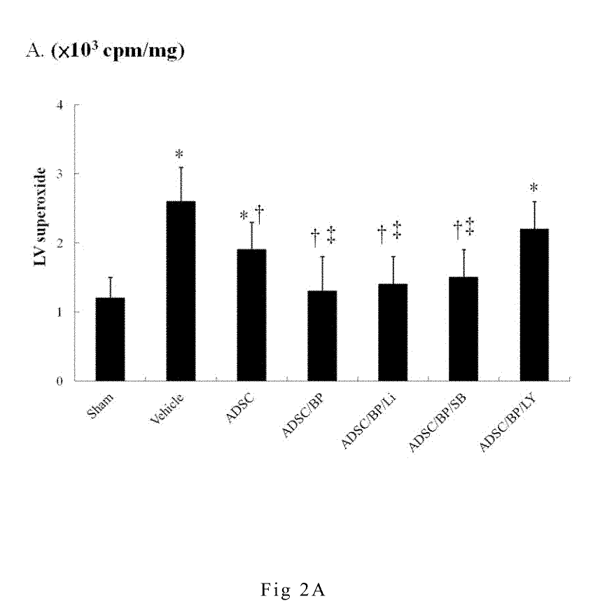

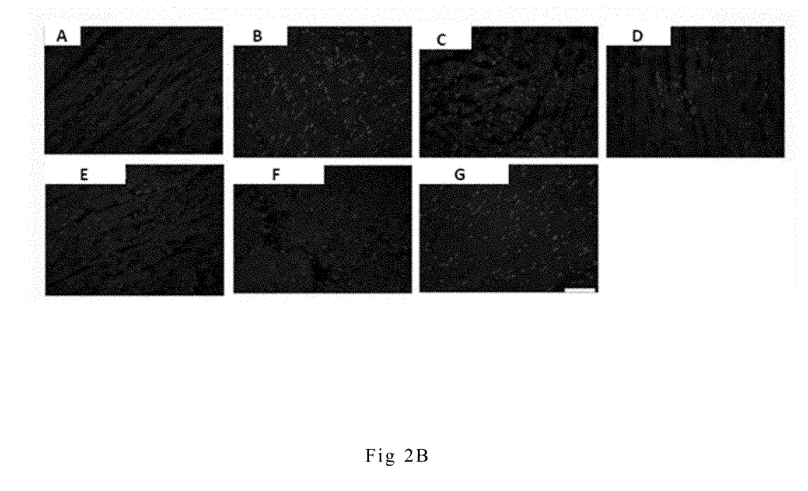

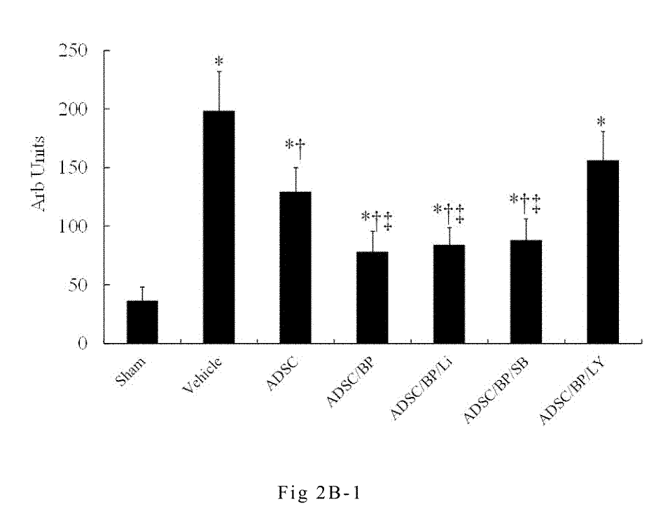

Myocardial superoxide production, as assessed by lucigenin-enhanced chemiluminescence, was markedly increased in the remote zone after MI as compared with sham (P<0.001, FIG. 2A). Superoxide was significantly decreased in ADSC-treated rats compared with vehicle. The superoxide levels were further reduced after adding BP treatment. The BP enhances the therapeutic effect on the heart via the pathways of P13K and GSK-3β∘

DHE reacts with superoxide radicals to form ethidium bromide, which in turn intercalates with DNA to provide nuclear fluorescence as a marker of superoxide radical generation. As shown in FIG. 2B and FIG. 2B-1, postinfarction remodeling markedly enhanced the intensity of the DHE staining in the remote zone in the vehicle-treated infarcted rats compared with sham. However, the intensity of the fluorescent signal in the BP-treated group was significantly reduced relative to the ADSC group. The BP enhances the therapeutic effect on the heart via the pathways of P13K and GSK-3β∘

The decreased ROS levels assessed by myocardial superoxide production and DHE staining in the BP-treated group were substantially reversed after coadministration of LY294002.

Effects of BP-Pretreated ADSC Transplantation on Cardiac Sympathetic Innervation

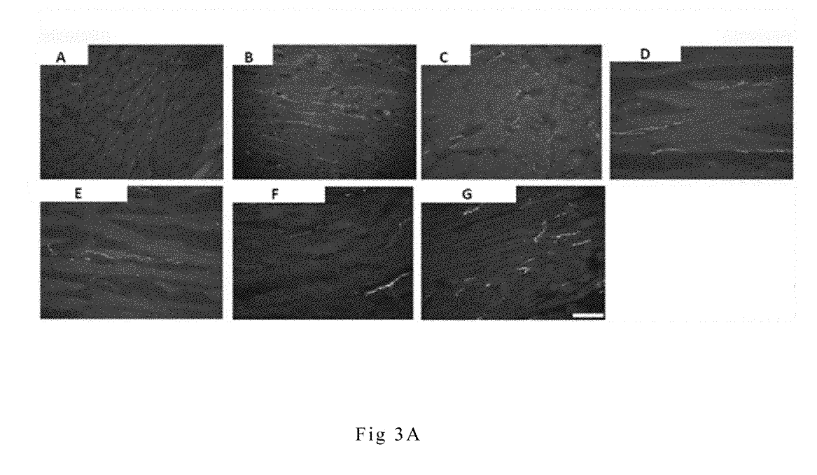

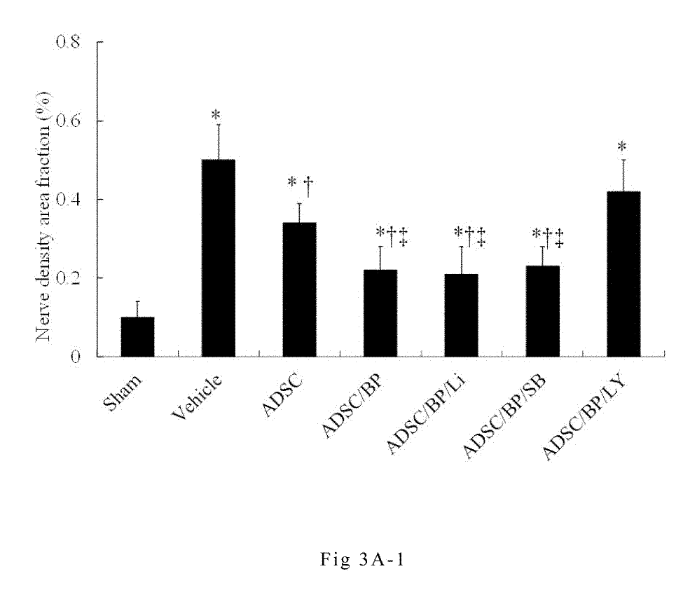

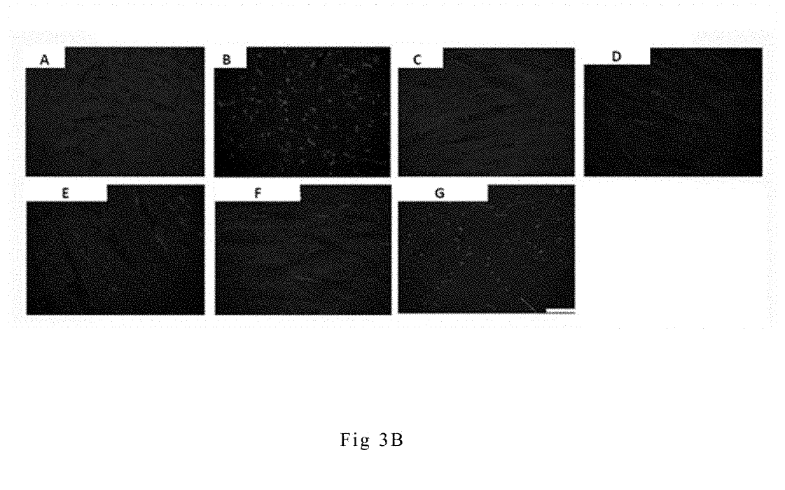

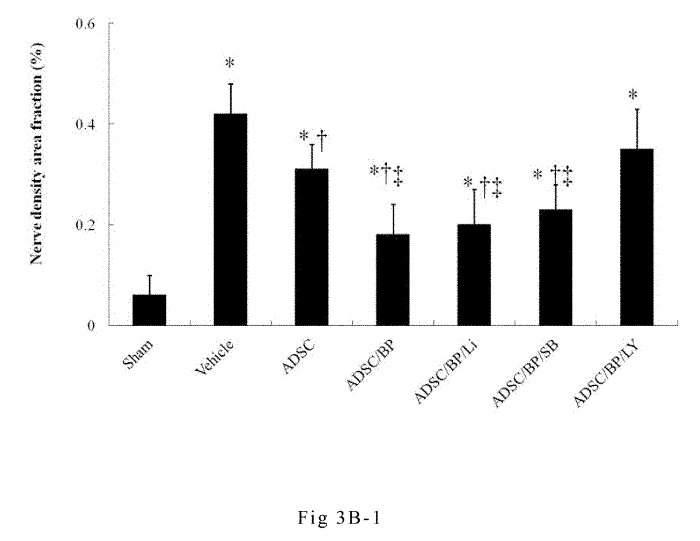

To investigate the cardiac sympathetic hyperinnervation after infarction, we determined the sympathetic nerve anatomy and function by analyzing immunofluorescent analyses and myocardial norepinephrine levels. The tyrosine hydroxylase-immunostained nerve fibers appeared to be oriented in the longitudinal axis of adjacent myofibers (FIGS. 3A, 3A-1). Tyrosine hydroxylase-positive nerve density was significantly increased in the vehicle-treated infarcted rats than that in sham. ADSC-treated rats showed significantly lower nerve density in the remote regions than vehicle-treated rats (0.37±0.04% in ADSC, vs 0.52±0.08% in vehicle, p<0.001, respectively). Similar to tyrosine hydroxylase results, densities of growth associated protein 43—(FIGS. 3B, 3B-1) and neurofilament-positive (data not shown) nerves were significantly attenuated in the BP-treated infarcted rats compared with those in ADSC-treated infarcted rats. However, the beneficial attenuated nerve density of BP was reversed after adding LY294002.

LV norepinephrine levels were significantly upregulated 2.35-fold in the remote zone in the vehicle-treated rats than in sham-operated rats (3.18±0.26 vs. 1.35±0.35 μg protein, p<0.0001, Table 1). Compared with ADSC-treated rats in BP-pretreated ADSC rats, LV norepinephrine levels were significantly further lower in the remote regions. The attenuated effect of BP on LV norepinephrine was reversed after adding LY294002. These functional study results mirrored those of immunofluorescent analyses.

Effects of BP-Pretreated ADSC Transplantation on Arrhythmias

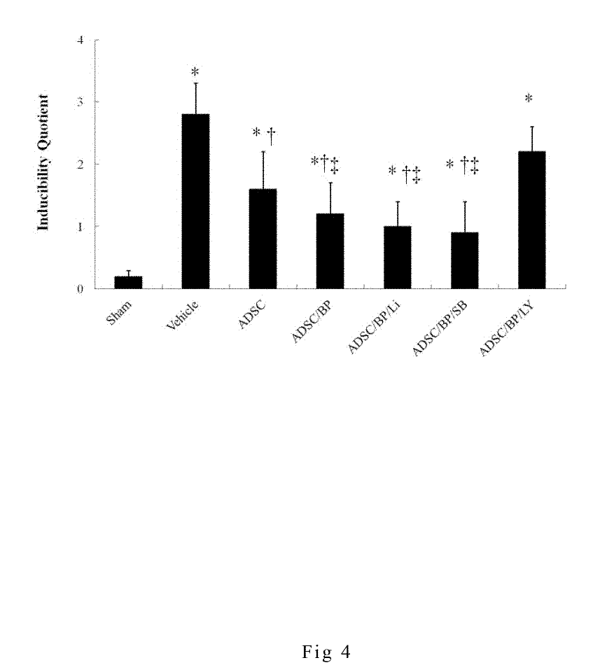

To further elucidate the physiological effect of attenuated sympathetic hyperinnervation, ventricular pacing was performed. Arrhythmia score in sham-operated rats was very low (0.2±0.1) (FIG. 4). In contrast, ventricular tachyarrhythmias consisting of ventricular tachycardia and ventricular fibrillation were inducible by programmed stimulation in vehicle-treated infarcted rats. ADSC treatment significantly decreased the inducibility of ventricular tachyarrhythmias compared with vehicle. BP pretreatment further decreased ventricular vulnerability compared with ADSC-treated alone. The addition of either lithium or SB216763 did not have additional beneficial effects compared with those seen in rats treated with BP alone. Conversely, the rats to which LY294002 was administered significantly increased the inducibility of ventricular tachyarrhythmias compared with the BP-treated rats alone.

TABLE 1
Cardiac morphometry, hemodynamics, and NE at the end of study

| | | Infarction treated with | | | | | |
|---|---|---|---|---|---|---|---|
| | | | | | | ADSC/BP/ | |
| Parameters | Sham | Vehicle | ADSC | ADSC/BP | ADSC/BP/Li | SB | ADSC/BP/LY |
| No. of rats | 7 | 5 | 5 | 5 | 5 | 5 | 5 |
| Mortality, n | 0 (0) | 3 (38%) | 4 (44%) | 3 (38%) | 3 (38%) | 4 (44%) | 4 (44%) |
| (%) | | | | | | | |
| Body weight, g | 313 ± 25 | 305 ± 22 | 312 ± 30 | 322 ± 21 | 313 ± 24 | 320 ± 20 | 311 ± 23 |
| HR, bpm | 388 ± 12 | 390 ± 15 | 401 ± 21 | 412 ± 20 | 402 ± 22 | 405 ± 15 | 411 ± 15 |
| LVESP, mm | 102 ± 4 | 95 ± 8 | 98 ± 6 | 101 ± 7 | 102 ± 7 | 101 ± 8 | 95 ± 8 |
| Hg | | | | | | | |
| LVEDP, mm | 6 ± 1 | 18 ± 2* | 16 ± 3* | 16 ± 3* | 17 ± 5* | 15 ± 4* | 20 ± 4* |
| Hg | | | | | | | |
| LVW/BW, | 2.15 ± 0.15 | 2.45 ± 0.24 | 2.46 ± 0.41 | 2.14 ± 0.20 | 2.22 ± 0.28 | 2.32 ± 0.31 | 2.52 ± 0.26 |
| mg/g | | | | | | | |
| RVW/BW, | 0.49 ± 0.11 | 0.84 ± 0.12* | 0.58 ± 0.12† | 0.52 ± 0.07† | 0.58 ± 0.15† | 0.62 ± 0.11† | 0.88 ± 0.21* |
| mg/g | | | | | | | |
| LungW/BW, | 4.28 ± 0.53 | 6.23 ± 0.57* | 5.17 ± 0.47*† | 4.98 ± 0.49† | 5.02 ± 0.48† | 4.89 ± 0.47† | 6.59 ± 0.44* |
| mg/g | | | | | | | |
| +dp/dt, mm | 7943 ± 315 | 2874 ± 347* | 3674 ± 315*† | 4132 ± 247*†‡ | 4278 ± 382*†‡ | 4345 ± 362*†‡ | 2984 ± 298* |
| Hg/sec | | | | | | | |
| −dp/dt, mm | 6945 ± 355 | 2487 ± 306* | 3283 ± 294*† | 3697 ± 279*†‡ | 3702 ± 241*†‡ | 3742 ± 275*†‡ | 2354 ± 292* |
| Hg/sec | | | | | | | |
| Infarct size, % | ... | 40 ± 2 | 38 ± 3 | 38 ± 3 | 38 ± 2 | 39 ± 4 | 40 ± 3 |
| NE, μg/g | 1.35 ± 0.35 | 3.18 ± 0.26* | 2.04 ± 0.29*† | 1.92 ± 0.32*† | 1.96 ± 0.32*† | 2.15 ± 0.44*† | 3.42 ± 0.36* |
| protein | | | | | | | |

Values are mean±SD. BP, z-butylidenephthalide; BW, body weight; HR, heart rate; LungW, lung weight; Li, lithium; LVEDP, left ventricular end-diastolic pressure; LVESP, left ventricular end-systolic pressure; LVW, left ventricular weight; LY, LY294002RVW, right ventricular weight; SB, SB216763. *p<0.005 compared with sham; †p<0.05 compared with infarcted groups treated with vehicle and ADSCs/BP/LY; ‡p<0.05 compared with infarcted group treated with ADSCs.

TABLE 2
Echocardiographic findings

| | | Infarction | | | | | |
|---|---|---|---|---|---|---|---|
| | Sham | treated with | | | | | |
| Parameters | Saline | Vehicle | ADSC | ADSC/BP | ADSC/BP/Li | ADSC/BP/SB | ADSC/BP/LY |
| Interventricular | 1.6 ± 0.2 | 0.7 ± 0.2* | 0.8 ± 0.2* | 0.8 ± 0.1* | 0.8 ± 0.3* | 0.8 ± 0.2* | 0.8 ± 0.2* |
| septum (mm) | | | | | | | |
| LVEDD (mm) | 5.9 ± 0.2 | 9.2 ± 0.2* | 7.5 ± 0.2*† | 7.4 ± 0.2*† | 7.7 ± 0.3*† | 7.5 ± 0.2*† | 8.8 ± 0.2* |
| LVESD (mm) | 3.7 ± 0.2 | 7.4 ± 0.2* | 5.7 ± 0.2*† | 5.2 ± 0.3*† | 5.4 ± 0.2*† | 5.4 ± 0.2*† | 7.3 ± 0.2* |
| LV posterior wall | 1.6 ± 0.1 | 1.8 ± 0.3* | 1.7 ± 0.1† | 1.7 ± 0.2 | 1.8 ± 0.2* | 1.8 ± 0.2* | 2.2 ± 0.1* |
| (mm) | | | | | | | |
| FS (%) | 37 ± 3 | 20 ± 4* | 24 ± 3*† | 30 ± 3*†‡ | 30 ± 3*†‡ | 28 ± 4*† | 17 ± 3* |

Effects of BP-Pretreated ADSC Transplantation on Akt Activity and NGF Expression

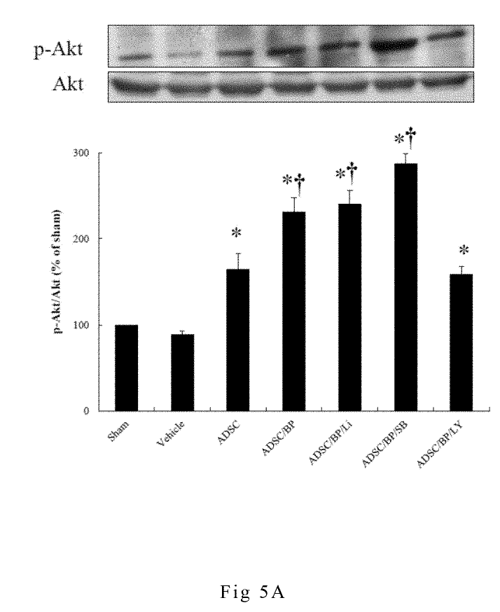

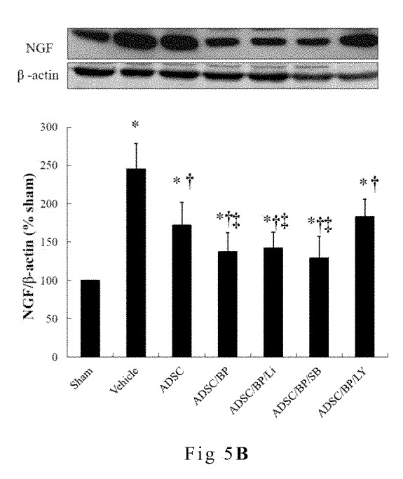

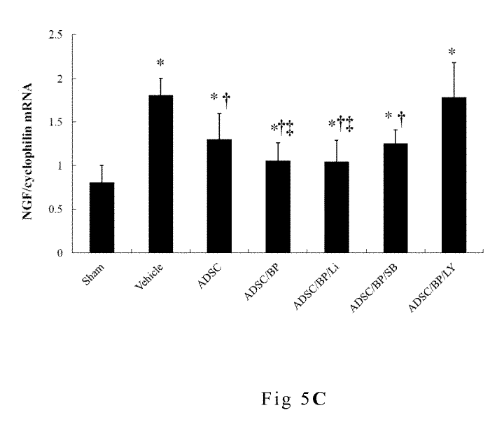

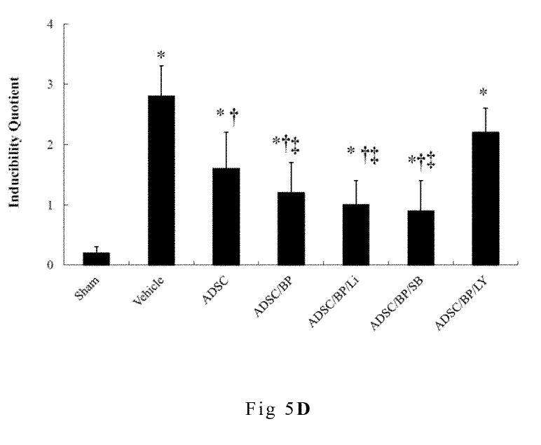

Western blot showed that BP primed ADSCs resulted in a significant increase (p<0.05) in relative p-Akt level compared with that in ADSCs alone (FIG. 5A). Western blot shows that NGF levels were significantly upregulated 2.45-fold in the remote zone in the vehicle-treated infarcted rats than in sham-operated rats (p<0.001, FIG. 5B). When compared with vehicle-treated infarcted rats, BP-pretreated infarcted rats had significantly lower NGF levels in the remote zone. PCR amplification of the cDNA revealed that the NGF mRNA levels showed a 2.25-fold upregulation in the remote zone in the vehicle-treated infarcted rats compared with sham-operated rats (p<0.001, FIG. 5C). n BP-pretreated ADSC rats, the NGF mRNA levels were significantly decreased compared with those in the ADSC-treated rats. Conversely, Akt activity and NGF expression in the LV was significantly reversed in rats treated with a combination of BP and LY294002 compared with BP-treated rats alone.

Effects of BP-Pretreated ADSC Transplantation on Arrhythmias

To further elucidate the physiological effect of attenuated sympathetic hyperinnervation, ventricular pacing was performed. Arrhythmia score in sham-operated rats was very low (0.2±0.1) (FIG. 5D). In contrast, ventricular tachyarrhythmias consisting of ventricular tachycardia and ventricular fibrillation were inducible by programmed stimulation in vehicle-treated infarcted rats. ADSC treatment significantly decreased the inducibility of ventricular tachyarrhythmias compared with vehicle. BP pretreatment further decreased ventricular vulnerability compared with ADSC-treated alone. The addition of either lithium or SB216763 did not have additional beneficial effects compared with those seen in rats treated with BP alone. Conversely, the rats to which LY294002 was administered significantly increased the inducibility of ventricular tachyarrhythmias compared with the BP-treated rats alone.

What is claimed is:

1. A pharmaceutical composition for therapeutic arrhythmia, comprising:
a stem cell and an inhibitor, wherein the stem cell is pretreat with a n-butylidenephthalide (BP).

2. The pharmaceutical composition according to claim 1,wherein the inhibitor is a GSK-3β inhibitor or PI3K inhibitor.

3. The pharmaceutical composition according to claim 2, wherein the GSK-3β inhibitor or the PI3K inhibitor is Lithium.

4. The pharmaceutical composition according to claim 2, wherein the GSK-3β inhibitor is SB216763.

5. The pharmaceutical composition according to claim 1, wherein the stem cell is an adipose-derived stem cell.

6. The pharmaceutical composition according to claim 1, wherein the n-butylidenephthalide (BP) concenration range is from 7 μg/ml to 40 μg/ml.

7. The pharmaceutical composition according to claim 1, wherein the GSK-3β (LITHIUM) concenration range is from 0.3 mM to 3 mM.

8. The pharmaceutical composition according to claim 1, wherein the PI3K inhibitor concenration range is 20 μM for LY294002.

9. A method for therapeutic arrhythmia in a subject, wherein the method comprising administering to said subject an effective amount of a pharmaceutical composition as claim 1.

10. The method according to claim 9, wherein the effective amount concentration range is from 1×10⁶ to 1×10⁷ cells.

11. The method according to claim 9, wherein the pharmaceutical composition is administering to said subject by intra-myocardial [symbol] intra-cardiac [symbol] intra-coronary artery [symbol] intra-cornary vein [symbol] trans-endocardial [symbol] intravenous [symbol] intra-muscular•intra-dermal [symbol] sub-cutaneous [symbol] intra-peritioneal [symbol] intra-pleural [symbol] systemic injection.

12. The method according to claim 9, wherein the arrhythmia is cause by Myocardial infarction.


SEQUENCE LISTING (6 sequences)

SEQ ID NO: 1 - DNA, 21 bp, Artificial Sequence
  human Alu sense primer
catggtgaaaccccgtctcta

SEQ ID NO: 2 - DNA, 19 bp, Artificial Sequence
  human Alu antisense primer
gcctcagcctcccgagtag

SEQ ID NO: 3 - DNA, 20 bp, Artificial Sequence
  NGF sense primer
cacactgaggtgcatagcgt

SEQ ID NO: 4 - DNA, 20 bp, Artificial Sequence
  NGF antisense primer
tgatgaccgcttgctcctgt

SEQ ID NO: 5 - DNA, 25 bp, Artificial Sequence
  cyclophilin sense primer
atggtcaaccccaccgtgttcttcg

SEQ ID NO: 6 - DNA, 25 bp, Artificial Sequence
  cyclophilin antisense primer
cgtgtgaagtcaccaccctgacaca